(19) United States
(12) Patent Application Publication
(10) Pub. No.: US 2017/0293942 A1
(43) Pub. Date: Oct. 12, 2017

(54) METHOD AND APPARATUS FOR PRESENTING ADVERTISEMENTS IN A VIRTUALIZED ENVIRONMENT

(71) Applicant: R-STOR INC., Saratoga, CA (US)

(72) Inventor: Giovanni COGLITORE, Saratoga, CA (US)

(21) Appl. No.: 15/485,882

(22) Filed: Apr. 12, 2017

Related U.S. Application Data
(60) Provisional application No. 62/321,534, filed on Apr. 12, 2016.

(51) Int. Cl. G06Q 30/02; A61B 5/16; A61B 5/0476; H04L 29/08; G06F 3/01

(52) U.S. Cl. CPC G06Q 30/0269; H04L 67/306; G06Q 30/0242; G06F 3/013; A61B 5/0476; A61B 5/165; A61B 2503/12

(57) ABSTRACT

Certain embodiments may relate to presenting advertisements in a virtualized environment. A method and apparatus may be provided. The method may include generating, by a virtual reality device, a virtualized environment for a user. The method may also include inserting an advertisement within the virtualized environment. The method may further include determining that the user has viewed the inserted advertisement. The method may also include determining a response of the user when the user views the inserted advertisement. The method may further include storing the response of the user.

### CROSS REFERENCE TO RELATED APPLICATION

This application claims priority to U.S. Provisional Application No. 62/321,534 filed on Apr. 12, 2016. The entire contents of the above-referenced provisional application in hereby incorporated by reference.

### BACKGROUND

### Field

Certain embodiments of the present invention may relate to presenting advertisements in a virtualized environment.

### Description of the Related Art

Virtual reality may generate/simulate a physical environment. Users of the virtual reality may interact within the simulated virtualized environment. The imagery of the virtualized environment may he displayed via a computer screen or via a headset. The virtualized environment may resemble physical locations in the real world or may resemble locations of an imaginary world.

### SUMMARY

According to a first embodiment, a method may include generating, by a virtual reality device, a virtualized environment for a user. The method may also include inserting an advertisement within the virtualized environment. The method may also include determining that the user has viewed the inserted advertisement. The method may also include determining a response of the user when the user views the inserted advertisement. The method may also include storing the response of the user.

In the method of the first embodiment, inserting the advertisement may include inserting a personalized advertisement, the personalized advertisement comprises an advertisement that is inserted based upon a profile of the user, and the profile of the user reflects characteristics of the user.

In the method of the first embodiment, the determining that the user has viewed the inserted advertisement comprises tracking the gaze of the user by the virtual reality device, and determining that the gaze has fallen on the inserted advertisement.

In the method of the first embodiment, the determining the response comprises determining an emotional or neurological response of the user, and the emotional or neurological response of the user is determined via brainwave measurements.

In the method of the first embodiment, the storing the response of the user comprises storing the response in accordance with the profile of the user.

According to a second embodiment, an apparatus may include at least one processor. The apparatus may also include at least one memory including computer program code. The at least one memory and the computer program code may be configured, with the at least one processor, to cause the apparatus at least to generate a virtualized environment for a user. The apparatus may also be caused to insert an advertisement within the virtualized environment. The apparatus may also be caused to determine that the user has viewed the inserted advertisement. The apparatus may also be caused to determine a response of the user when the user views the inserted advertisement. The apparatus may also be caused to store the response of the user.

In the apparatus of the second embodiment, inserting the advertisement comprises inserting a personalized advertisement, the personalized advertisement comprises an advertisement that is inserted based upon a profile of the user, and the profile of the user reflects characteristics of the user.

In the apparatus of the second embodiment, the determining that the user has viewed the inserted advertisement comprises tracking the gaze of the user by the virtual reality device, and determining that the gaze has fallen on the inserted advertisement.

In the apparatus of the second embodiment, the determining the response comprises determining an emotional or neurological response of the user, and the emotional or neurological response of the user is determined via brainwave measurements.

In the apparatus of the second embodiment, the storing the response of the user comprises storing the response in accordance with the profile of the user.

According to a third embodiment, a computer program product may be embodied on a non-transitory computer readable medium. The computer program product may be configured to control a processor to perform a method according to the first embodiment.

### BRIEF DESCRIPTION OF THE DRAWINGS

For proper understanding of the invention, reference should be made to the accompanying drawings, wherein:

FIG. 1 illustrates an example virtualized environment, in accordance with certain embodiments of the present invention.

FIG. 2 illustrates an example system that generates a virtualized environment and presents advertising, in accordance with certain embodiments of the present invention.

FIG. 3 illustrates a flowchart of a method in accordance with certain embodiments of the invention.

FIG. 4 illustrates an apparatus in accordance with certain embodiments of the invention.

FIG. 5 illustrates another apparatus in accordance with certain embodiments of the invention.

### DETAILED DESCRIPTION

Certain embodiments of the present invention may be directed to an advertising system/platform which inserts advertisements or advertised products within a virtualized environment. The inserted advertisements/products may be intended to be viewed by a user that is interacting within the virtualized environment.

The advertising system of certain embodiments may determine which advertisements/products should be inserted within the virtualized environment, based on the specific user within the virtualized environment. For example, certain characteristics of the specific user may trigger the insertion of a corresponding type of advertisement. For example, if the user is male, then certain embodiments may present advertisements that are directed to men. Alternatively, if the user is female, then certain embodiments may present advertisements that are directed to women. If the user is a teenager, then certain embodiments may present advertisements that are directed to teenagers.

The advertising system of certain embodiments may determine which advertisements/products should be inserted within the virtualized environment based on a generated user profile of a specific user. The generated user profile of the specific user may include information that indicates Which advertisements the specific user is likely to be interested in, is likely to react to, is likely to share with others, etc. In other words, the inserted advertisements may be personalized for the specific user in accordance with the user profile of the specific user, as described in more detail below.

Further, with certain embodiments, the advertising system may insert advertisements based on the type of virtualized environment. For example, if the virtualized environment is a sports arena, the inserted advertisements may be sports-related. If the virtualized environment is a theme park, the inserted advertisements may include souvenirs, memorabilia of the theme park, etc.

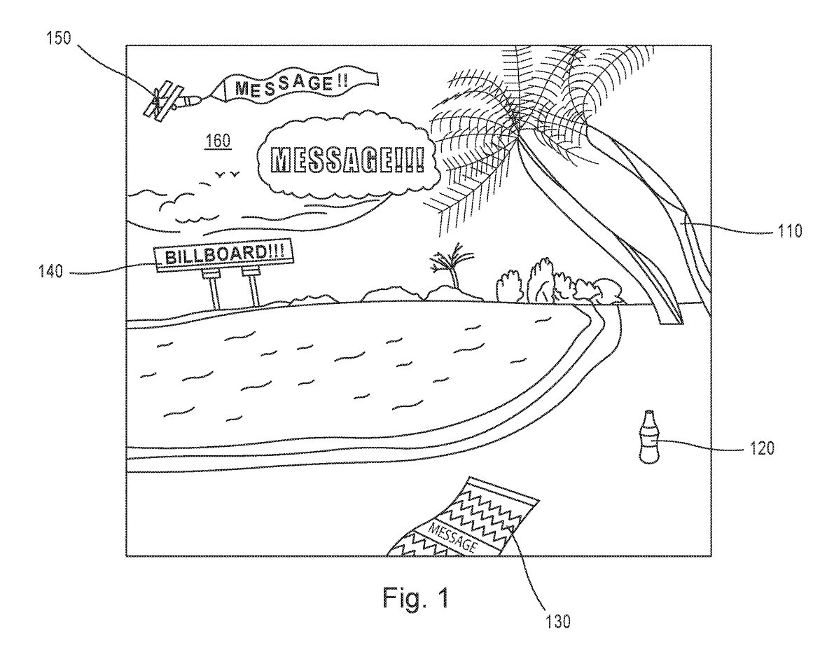

FIG. 1 illustrates an example virtualized environment 100, in accordance with certain embodiments of the present invention. As described above, a user may interact within the virtualized environment 100. The virtualized environment 100 may be a vacation destination, a ballpark, an imaginary destination, and/or any other environment that the user prefers. In the example of FIG. 1, the virtualized environment 100 may include objects such as palm trees 110, sand, waves, clouds, and other objects that complete the effect of the ⁻virtualized environment 100. As described above, certain embodiments may insert certain advertised products into the virtualized environment. For example, a beer or soft drink bottle 120 may be inserted into the virtualized environment. The type of product may be determined based on determined characteristics of the user. For example, for users under the age of 21, certain embodiments may insert soft drink bottles, rather than beer bottles.

In addition to inserting advertised products, certain embodiments may also insert advertisement messages within the virtualized environment. For example, in the example of FIG. 1, an advertisement message may appear on a beach towel 130, a billboard 140, an airplane banner 150, and within clouds 160, for example. In general, advertised messages and advertised products may be integrated within the virtualized environment in any manner, such that the advertised messages are a part of the virtualized environment. The advertised messages/products may be determined based on the selected virtualized environment (such as beach towel 130 for a beach environment), personalized based on a determined characteristic of the user (such as beer bottle 120 for users over the age of 21), or personalized based on a profile determined for the user, as described in more detail below.

The inserted advertisement within the virtualized environment 100 may be seamlessly integrated within the virtualized environment itself or may be an object within the virtualized environment. In addition to the examples shown in FIG. 1, an advertisement that is integrated within the environment itself may include, for example, a sign on a wall of the virtualized environment, a storefront within the virtualized environment, a message on a structure of the virtualized environment, etc. An advertisement that appears as an object within the environment may include a vehicle that is moving within the environment, different goods that are within the virtualized environment, flyers within the environment, etc. Advertisements may be integrated into the structure, objects, and persons of the virtualized environment.

With certain embodiments, in order to interact with the virtualized environment, the user of the virtualized environment may be utilizing hardware which simulates/generates the virtualized environment for the user. For example, the user may be wearing a virtual-reality headset or eyewear that generates the virtualized environment. The user may also use a projection hardware that simulates/generates the virtualized environment.

In any event, the hardware which generates the virtualized environment for the user may operate in conjunction with the advertising system/platform that inserts advertising within the virtualized environment. Therefore, as the virtualized environment is generated, the advertising system inserts advertisements and/or advertised products within the generated environment.

In addition to generating the virtualized environment, the hardware may also track the gaze of the user, in order to be able to determine what the user is looking at, at each particular point in time. For example, with certain embodiments, the hardware may track the eye movements of the user as the user interacts within the virtualized environment in order to determine what the user is looking at within the virtualized environment, at any given time. Certain embodiments may utilize an eye tracking device where the device is in contact with the user's eye (i.e., such as a contact lens that can track the user's eye movements). Other embodiments may use an eye tracking device that measures eye movements without directly contacting the user's eyes. Other embodiments may measure eye movements by measuring electrical signals of nerves at or around the user's eves.

Therefore, with certain embodiments, the hardware may track and record what the user is looking at during each moment when the user is interacting within the virtualized environment.

With certain embodiments, the hardware may operate in conjunction with the advertising system. By tracking the gaze of the viewer, the hardware may inform/indicate to the advertising system the instances where the user is viewing an advertisement within the virtualized environment. The hardware may then inform the advertising platform which advertisements that the user has viewed, how often the viewer views each advertisement, how long each advertisement is viewed, and/or the surrounding circumstances which caused the viewer to view each advertisement. By analyzing which advertisements are viewed, how often each advertisement is viewed, how long each advertisement is viewed, and/or the surrounding circumstances, the advertising system may begin to make determinations regarding which advertisements may be effective for a given user. For example, if an advertisement is viewed often, the system may determine that the given user is responding positively to the advertisement. Further, if an advertisement is viewed for a long duration of time, the system may determine that the given user is interested in the advertisement.

In addition to making determinations regarding the effectiveness of advertisements based on viewing patterns, certain embodiments may also determine the effectiveness of the advertisements based on emotional and/or neurological responses of the users, as the users view the inserted advertisements, as described in more detail below.

Certain embodiments of the present invention may measure the emotional and/or neurological responses of the user as the user interacts with the virtualized environment. For example, certain embodiments may include sensors that measure the brainwaves of the user as the user interacts with the virtualized environment. From the measured brainwaves, certain embodiments of the present invention may make determinations regarding the emotional or neurological state of the user at each particular moment.

With certain embodiments, the sensors may be integrated with the headset that generates the virtualized environment. Therefore, as the user puts on the headset to enter the virtualized environment, the user may also position the user's head in a position such that the user's neurological and/or emotional responses may be measured. For example, the brain waves of the user may be measured. Further, the sensors may also determine the intensity of a given response.

Therefore, with certain embodiments, as the advertising system tracks what the user is viewing at each particular instance, certain embodiments may determine the user's emotional/neurological response at each particular instance as well. Therefore, with certain embodiments, the advertising system may determine the user's emotional/neurological responses each time the viewer views different advertisements.

Therefore, as the advertising system determines a user's emotional or neurological response when viewing different advertisements, the advertising platform may thus more accurately gauge a user's reaction to an advertisement. For example, the advertising system may more accurately determine whether a user is interested in or disinterested in an advertisement.

As the user continues to interact within the virtualized environment, and continues to provide responses to advertisements (where these responses are recorded and stored by the advertising platform), the advertising platform may generate a profile of the user. The profile of the user will comprise information that indicates how the user responds to different advertisements or to different types of advertisements. For example, the profile of the user may indicate which advertisements are liked or disliked by the user. With certain embodiments, the profile of the user may indicate whether certain advertisements generate feelings of happiness, calm, excitement, sadness, anxiousness, nostalgia, regret, disgust, anger, desire, insecurity, pride, and/or fear. The profile of the user may also indicate the intensity of the user's response.

After a profile is generated for a user, the advertising platform may present advertisements in accordance with the profile. Therefore, the advertising platform may provide advertisements that more effectively elicit the desired response within the user, where the desired response is determined by the advertising system.

The advertising system may also present the collected data via an interface or print out to be viewed by operators of the advertising system. By viewing the collected data on the interface or print out, operators of the advertising system may more readily gauge which advertisements are effective, or ineffective

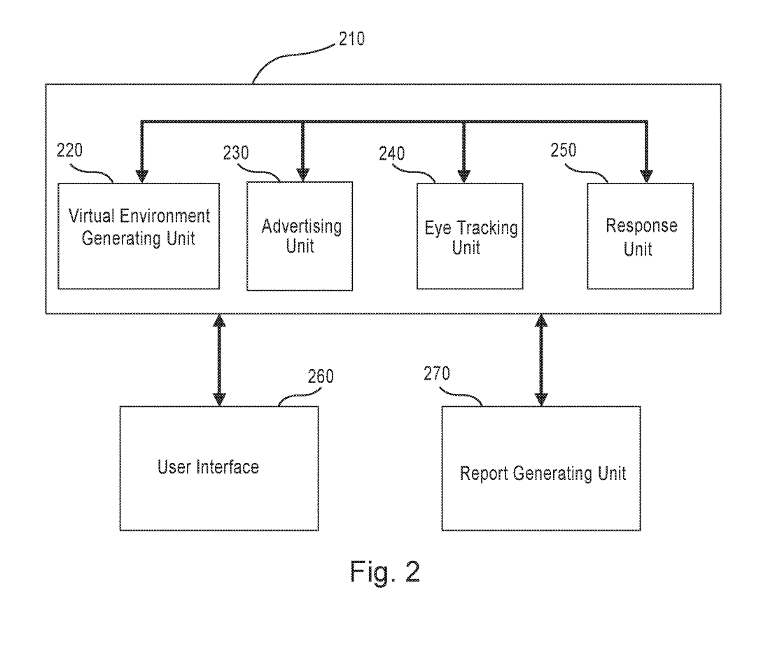

FIG. 2 illustrates an example system 210 that generates a virtualized environment and presents advertising, in accordance with certain embodiments of the present invention. The units of example system 210 may be integrated within a virtual reality headset. Alternatively, certain units of example system 210 may be separate from the virtual reality headset. The system 210 of FIG. 2 may include a virtual environment generating unit 220 that generates a virtualized environment, an eye tracking unit 240 that tracks the gaze of the user, a response unit 250 (such as, for example, sensors) that measures a neurological response, and an advertising unit/platform 230, in accordance with certain embodiments of the invention.

Referring to FIG. 2, as described above, a user may view an inserted advertisement that is inserted by advertising unit 230 within the virtual environment that is generated by virtual environment generating unit 220. After the user views the inserted advertisement, the eye tracking unit 240 may detect/determine that the user has viewed the inserted advertisement. The sensors of the response unit 250 may then measure the emotional/neurological response of the user in response to the viewed advertisement. The measured response may be stored by the advertising unit 230. The stored responses may then generate the profile of the given user. The advertising unit 230 may then subsequently insert future advertisements based on the updated profile of the given user.

Further, certain embodiments of the present invention may also include a user interface 260 that allows the advertising unit 230 to display data regarding the user profiles, the advertising trends, and/or any other results relating to advertising. The data may be displayed via interface 260 to operators or advertising professionals. Certain embodiments may also include a report generating unit 270 that generates reports to be viewed by the operators or advertising professionals. The reports may relate to the user profiles, the advertising trends, and/or any other results relating to advertising.

In view of the above, certain embodiments of the present invention may be able to provide advertisers with new opportunities to present their advertisements. Compared to traditional advertisements (that are presented via television or radio), certain embodiments may insert advertisements that are personalized to the intended viewer. Certain embodiments may also provide advertisers with clearer indications regarding which advertisements are effective, the features which make effective advertisements, and how viewers interact with the inserted advertisements.

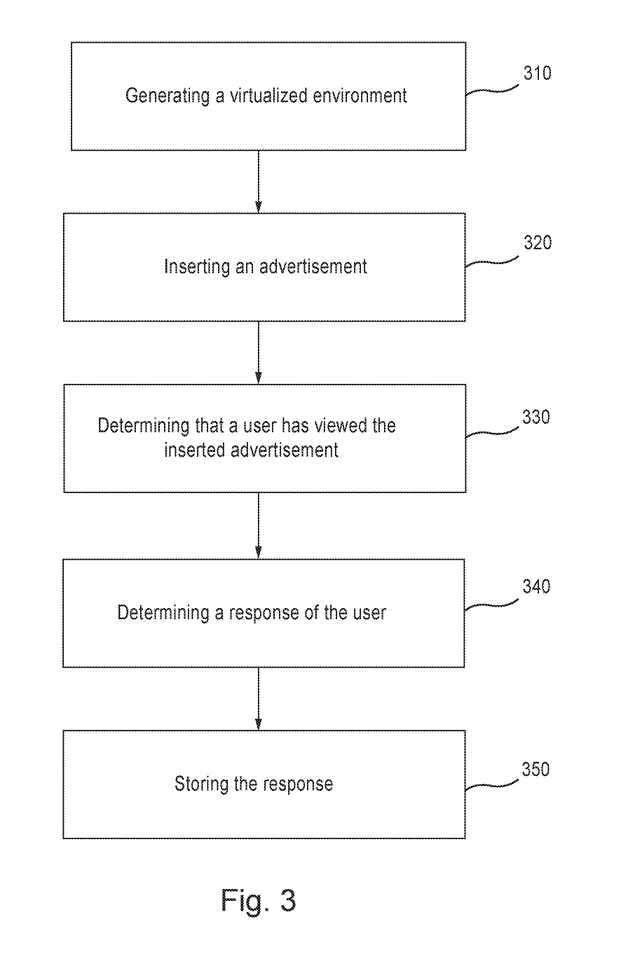

FIG. 3 illustrates a flowchart of a method in accordance with certain embodiments of the invention. The method illustrated in FIG. 3 includes, at 310, generating, by a virtual reality device, a virtualized environment for a user. The method may also include, at 320, inserting an advertisement within the virtualized environment. The method may also include, at 330 determining that the user has viewed the inserted advertisement. The method may also include, at 340, determining a response of the user when the user views the inserted advertisement. The method may also include, at 350, storing the response of the user.

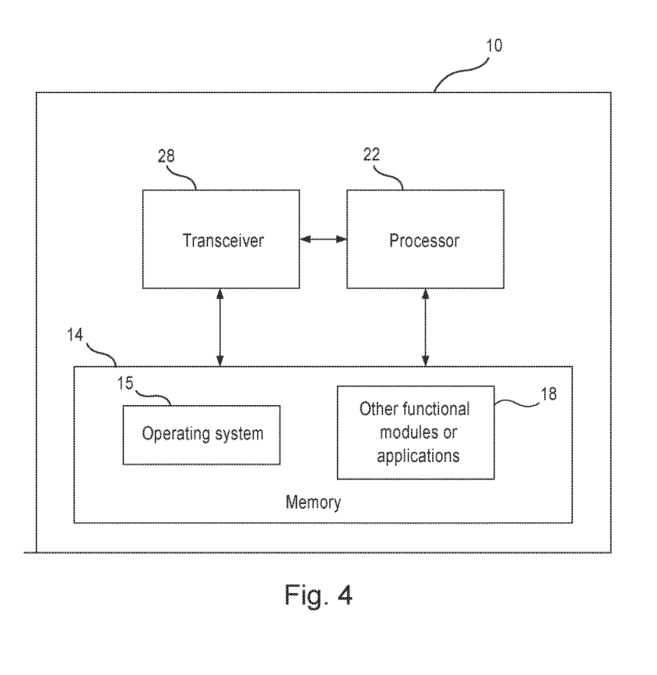

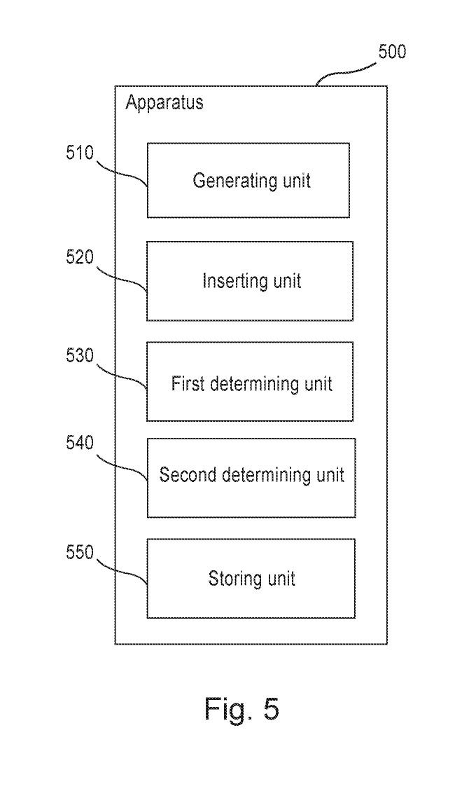

FIG. 4 illustrates an apparatus 10 according to certain embodiments of the present invention. It should be understood that each block in FIGS. 2 and 3 may be implemented by various means or their combinations, such as hardware, software, firmware, one or more processors and/or circuitry. In an embodiment, apparatus 10 may be a device that generates the virtualized environment (such as a headset or some other virtual reality device), for example. In another embodiment, apparatus 10 may be a device that implements functions of the advertising system which may be separate from the device that generates the virtualized environment. Apparatus 10 may also be configured to operate as any of environment generating unit 220, advertising unit 230, eye tracking unit 240, response unit 250, user interface 260, and/or report generating unit 270, as shown in FIG. 2. In addition, apparatus 10 may be configured to operate as any of the components of the apparatus 500, as shown in FIG. 5. For example, this may include the generating unit 510, inserting unit 520, first determining unit 530, second determining unit 540, and storing unit 550. Although only one apparatus 10 is shown in FIG. 4, more than one apparatus 10 may be implemented together in various combinations as a system, or independently.

Apparatus 10 includes a processor 22 for processing information and executing instructions or operations. Processor 22 may be any type of general or specific purpose processor. While a single processor 22 is shown in FIG. 4, multiple processors may be utilized according to other embodiments. In fact, processor 22 may include one or more of general-purpose computers, special purpose computers, microprocessors, digital signal processors (“DSPs”), field-programmable gate arrays (“FPGAs”), application-specific integrated circuits (“ASICs”), and processors based on a multi-core processor architecture, as examples.

Apparatus 10 further includes a memory 14, coupled to processor 22, for storing information and instructions that may be executed by processor 22. Memory 14 may be one or more memories and of any type suitable to the local application environment, and may be implemented using any suitable volatile or nonvolatile data storage technology such as a semiconductor-based memory device, a magnetic memory device and system, an optical memory device and system, fixed memory, and removable memory. For example, memory 14 can be comprised of any combination of random access memory (“RAM”), read only memory (“ROM”), static storage such as a magnetic or optical disk, or any other type of non-transitory machine or computer readable media. The instructions stored in memory 14 may include program instructions or computer program code that, when executed by processor 22, enable the apparatus 10 to perform tasks as described herein, including that illustrated in FIG. 3.

Apparatus 10 may also include one or more antennas (not shown) for transmitting and receiving signals and/or data to and from apparatus 10. Apparatus 10 may further include a transceiver 28 that modulates information on to a carrier waveform for transmission by the antenna(s) and demodulates information received via the antenna(s) for further processing by other elements of apparatus 10. In other embodiments, transceiver 28 may be capable of transmitting and receiving signals or data directly.

Processor 22 may perform functions associated with the operation of apparatus 10 including, without limitation, preceding of antenna gain/phase parameters, encoding and decoding of individual bits forming a communication message, formatting of information, and overall control of the apparatus 10, including processes related to management of communication resources.

In an embodiment, memory 14 stores software modules that provide functionality when executed by processor 22. The modules may include an operating system 15 that provides operating system functionality for apparatus 10. The memory may also store one or more functional modules 18, such as an application or program, to provide additional functionality for apparatus 10. The components of apparatus 10 may he implemented in hardware, or as any suitable combination of hardware and software.

In one embodiment, apparatus 10 may be configured to generate a virtualized environment to a user. Apparatus 10 may also be configured to insert an advertisement within the virtualized environment. Apparatus 10 may also he configured to determine that the user has viewed the inserted advertisement. Apparatus 10 may also be configured to determine a response of the user when the user views the inserted advertisement. Apparatus 10 may also be configured to store the response of the user.

FIG. 5 illustrates another apparatus in accordance with certain embodiments of the invention. Apparatus 500 can be a headset or some other virtual reality device, for example. Apparatus 500 can include a generating unit 510 that generates a virtualized environment to a user. Apparatus 500 may also include an inserting unit 520 that inserts an advertisement within the virtualized environment. Apparatus 500 may also include a first determining unit 530 that determines that the user has viewed the inserted advertisement. Apparatus 500 may also include a second determining unit 540 that determines a response of the user when the user views the inserted advertisement. Apparatus 500 may also include a storing unit 550 that stores the response of the user.

The described features, advantages, and characteristics of the invention can be combined in any suitable manner in one or more embodiments. One skilled in the relevant art will recognize that the invention can be practiced without one or more of the specific features or advantages of a particular embodiment. In other instances, additional features and advantages can be recognized in certain embodiments that may not be present in all embodiments of the invention. One having ordinary skill in the art will readily understand that the invention as discussed above may be practiced with steps in a different order, and/or with hardware elements in configurations which are different than those which are disclosed. Therefore, although the invention has been described based upon these preferred embodiments, it would be apparent to those of skill in the art that certain modifications, variations, and alternative constructions would be apparent, while remaining within the spirit and scope of the invention.

We claim:

1. A method, comprising:
generating, by a virtual reality device, a virtualized environment for a user;
inserting an advertisement within the virtualized environment;
determining that the user has viewed the inserted advertisement;
determining a response of the user when the user views the inserted advertisement; and
storing the response of the user.

2. The method of claim 1,
wherein the inserting the advertisement comprises inserting a personalized advertisement,
wherein the personalized advertisement comprises an advertisement that is inserted based on a profile of the user, and the profile of the user reflects characteristics of the user.

3. The method of claim 1, wherein the determining that the user has viewed the inserted advertisement comprises tracking a gaze of the user by the virtual reality device, and determining that the gaze has fallen on the inserted advertisement.

4. The method of claim 1,
wherein the determining the response comprises determining an emotional or neurological response of the user, and
wherein the emotional or neurological response of the user is determined via brainwave measurements.

5. The method of claim 2, wherein the storing the response of the user comprises storing the response in accordance with the profile of the user.

6. An apparatus, comprising:
at least one processor; and
at least one memory including computer program code,
wherein the at least one memory and the computer program code are configured to, with the at least one processor, cause the apparatus at least to:
generate a virtualized environment for a user;
insert an advertisement within the virtualized environment;
determine that the user has viewed the inserted advertisement;
determine a response of the user when the user views the inserted advertisement; and
store the response of the user.

7. The apparatus of claim 6,
wherein inserting the advertisement comprises inserting a personalized advertisement,
wherein the personalized advertisement comprises an advertisement that is inserted based on a profile of the user, and the profile of the user reflects characteristics of the user.

8. The apparatus of claim 6, wherein the determining that the user has viewed the inserted advertisement comprises tracking a gaze of the user by the virtual reality device, and determining that the gaze has fallen on the inserted advertisement.

9. The apparatus of claim 6,
wherein the determining the response comprises determining an emotional or neurological response of the user, and
wherein the emotional or neurological response of the user is determined via brainwave measurements.

10. The apparatus of claim 7, wherein the storing the response of the user comprises storing the response in accordance with the profile of the user.

11. A computer program, embodied on a non-transitory computer-readable medium, the computer program, when executed by a processor, causes the processor to:
generate a virtualized environment for a user;
insert an advertisement within the virtualized environment;
determine that the user has viewed the inserted advertisement;
determine a response of the user when the user views the inserted advertisement; and
store the response of the user.

12. The computer program of claim 11,
wherein inserting the advertisement comprises inserting a personalized advertisement,
wherein the personalized advertisement comprises an advertisement that is inserted based on a profile of the user, and the profile of the user reflects characteristics of the user.

13. The computer program of claim 11, wherein the determining that the user has viewed the inserted advertisement comprises tracking a gaze of the user by the virtual reality device, and determining that the gaze has fallen on the inserted advertisement.

14. The computer program of claim 11,
wherein the determining the response comprises determining an emotional or neurological response of the user, and
wherein the emotional or neurological response of the user is determined via brainwave measurements.

15. The computer program of claim 12, wherein the storing the response of the user comprises storing the response in accordance with the profile of the user.
